(19) United States
(12) Patent Application Publication
(10) Pub. No.: US 2005/0255569 A1
(43) Pub. Date: Nov. 17, 2005

(54) Method for producing triterpene composition

(75) Inventors: Futoshi Matsuyama, Fukuchiyama-shi (JP); Takashi Yoshida, Okayama-shi (JP); Tsutomu Hatano, Okayama-shi (JP); Shoko Taniguchi, Okayama-shi (JP)

Correspondence Address: MORGAN LEWIS & BOCKIUS LLP, 1111 PENNSYLVANIA AVENUE NW, WASHINGTON, DC, 20004, US

(73) Assignee: USE-TECHNO CORPORATION

(21) Appl. No.: 11/020,577

(22) Filed: Dec. 27, 2004

Related U.S. Application Data
(60) Provisional application No. 60/532,578, filed on Dec. 29, 2003.

(30) Foreign Application Priority Data
Dec. 26, 2003 (JP) P2003-433867

(51) Int. Cl. C12P 15/00; C12N 5/04

(52) U.S. Cl. 435/127; 435/419

(57) ABSTRACT

The present invention provides a method of producing a triterpene composition wherein callus induced from banaba, loquat, perilla or guava is cultured with indoleacetic acid and another plant hormone, and a triterpene composition containing at least one selected from the group consisting of corosolic acid, tormentic acid, 2α,19α-dihydroxy-3-oxo-urs-12-en-28-oic acid and maslinic acid is obtained from the cultured callus.

### CROSS-REFERENCE TO RELATED APPLICATION

This application claims priority to Provisional Application Ser. No. 60/532,578 filed on Dec. 29, 2003, which is hereby incorporated by reference in its entirety.

### BACKGROUND OF THE INVENTION

1. Field of the Invention

The present invention relates to a method of producing a triterpene composition, to a triterpene composition, and to callus used to produce a triterpene composition.

2. Related Background Art

It is known that triterpenes, such as corosolic acid, tormentic acid and maslinic acid, are useful substances each having pharmacological actions such as a blood glucose depressant action, and examples wherein these triterpenes were obtained as ingredients extracted from plants have already been reported.

An example of a plant containing corosolic acid is banaba (Lagerstroemia speciosa), (e.g., “Japanese Pharmacology and Therapeutics”, 1999, Vol. 27, No. 6, pp. 1075-1077), an example of a plant containing tormentic acid is Tiarella polyphyla (e.g., Archives of Pharmacal Research, Korea, 2002, Vol. 25, No. 1, pp. 57-60), and an example of a plant containing maslinic acid is the olive (Olea europasea) (e.g., International Publication No. WO 03/057224).

### SUMMARY OF THE INVENTION

However, the triterpene content of plants was not necessarily very high, and triterpenes could not necessarily be obtained efficiently. If the plants were grown artificially, cultivation required a period of several weeks or more, and since the triterpene content was easily affected by climate and the soil used to culture them, there was also a problem in respect of stability of supply.

Moreover, no technique was known for performing the chemosynthesis of triterpenes such as corosolic acid, tormentic acid, 2α,19α-dihydroxy-3-oxo-urs-12-en-28-oic acid and maslinic acid economically.

It is therefore an object of the present invention to provide a means of stably and efficiently supplying a triterpene composition containing at least one triterpene selected from the group consisting of corosolic acid, tormentic acid, 2α,19α-dihydroxy-3-oxo-urs-12-en-28-oic acid and maslinic acid.

Callus culture is a technique of cultivating callus induced by cultivating parts taken from plants such as leaves, stems and roots on a culture medium, and may be used for basic research into the biosynthesis of metabolites.

The present inventors cultured callus with a specific plant hormone in this callus culture using banaba, loquat, perilla or guava as a plant from which callus was induced. They discovered that specific triterpenes could be produced very efficiently in the cultured callus, and thus arrived at the present invention.

Specifically, a method of producing a triterpene composition provided by the present invention is a method wherein callus induced from banaba, loquat, perilla or guava is cultured with indoleacetic acid and another plant hormone, and a triterpene composition containing at least one triterpene selected from the group consisting of corosolic acid, tormentic acid, 2α,19α-dihydroxy-3-oxo-urs-12-en-28-oic acid and maslinic acid is obtained from the cultured callus.

In the method of producing a triterpene composition of the present invention, it is preferred to carry out the subculturing of callus. By carrying out the subculturing of callus, the triterpene composition can be continuously obtained from the callus induced from banaba etc., without newly inducing another callus.

The callus can be induced from leaves, seeds, stems, shoot tips, roots or germ cells. The triterpene composition can be obtained efficiently in a shorter period of time by inducing callus from these parts.

The cultured callus preferably contains maslinic acid, corosolic acid at 4-10 times by weight with respect to maslinic acid, tormentic acid at 2-8 times by weight with respect to maslinic acid, and 2α,19α-dihydroxy-3-oxo-urs-12-en-28-oic acid at 1.5-4 times by weight with respect to maslinic acid. To facilitate obtaining corosolic acid, which is very useful in lowering blood glucose level, in high purity, it is particularly important that the ratio of corosolic acid to maslinic acid is high.

The present invention further provides callus induced from banaba, loquat, perilla or guava which is used in the above producing method. The callus of the present invention induced from banaba, loquat, perilla or guava contains a large amount of triterpenes such as corosolic acid, tormentic acid, 2α,19α-dihydroxy-3-oxo-urs-12-en-28-oic acid and maslinic acid.

Therefore, a triterpene composition obtained by the above producing method containing at least one selected from the group consisting of corosolic acid, tormentic acid, 2α,19α-dihydroxy-3-oxo-urs-12-en-28-oic acid and maslinic acid can be provided. This triterpene composition can be used as an ingredient of health foods, medicines, cosmetics, and the like.

The present invention further provides a method of producing a triterpene composition whereby triterpenes such as corosolic acid, tormentic acid, 2α,19α-dihydroxy-3-oxo-urs-12-en-28-oic acid and maslinic acid can be each isolated and purified from this triterpene composition. By applying this producing method, triterpenes of high purity for use in pharmaceutical products can be obtained quite efficiently.

### DESCRIPTION OF THE PREFERRED EMBODIMENTS

Embodiments of the present invention will now be described in detail.

In the method of producing a triterpene composition of the present invention, callus (an undifferentiated cell cluster induced from plant cells) is induced from banaba, loquat, perilla or guava.

Among these, it is preferred to induce callus from banaba. Banaba (scientific name: Lagerstroemia speciosa, Linn. or Pers.) is a plant belonging to Lythraceae. It is used as a roadside tree, and its leaves are also used in traditional herbal medicine in the Philippines.

Banaba, loquat, perilla or guava which grows naturally in the wild or was grown artificially can be used in the induction of callus. To obtain a triterpene composition efficiently in a short time, callus is preferably induced from leaves, seeds, stems, shoot tips, roots or germ cells, but more preferably induced from leaves or seeds. The callus may be induced from protoplasts prepared from these parts.

The callus can be induced by, for example, sterilizing parts such as leaves cut from banaba with sodium hypochlorite etc., cutting them to a suitable size with a scalpel, transplanting them to a culture medium and cultivating them.

Although callus can be induced in a solid culture medium or a liquid culture medium, it is preferred to induce it in a solid culture medium such as a Linsmaier-Skoog agar medium (hereinafter referred to as an “LS agar medium”).

When the induced callus is cultured and proliferated in a culture medium to which indoleacetic acid and another plant hormone have been added, a large amount of triterpene is produced in the callus. In the culturing of callus induced from banaba, loquat, perilla or guava, it is particularly important to use indoleacetic acid and another plant hormone in combination. Compared to the case where only plant hormones other than indoleacetic acid are used, the efficiency of callus proliferation and triterpene production is remarkably improved.

Examples of plant hormones other than indoleacetic acid which can be used in combination with indoleacetic acid are kinetin, 1-naphthaleneacetic acid and 6-benzyladenine, among which kinetin is particularly preferred.

It is also preferred to culture the callus in a solid culture medium such as an LS agar medium under sterile conditions at 20-30° C. in a dark place.

The triterpene composition can be obtained directly from the cultured callus, but the triterpene composition can also be obtained by transplanting part of the cultured callus to another new solid culture medium or liquid culture medium and cultivating it again, i.e., by performing the subculturing of callus. By successively performing the subculturing, the triterpene composition can be stably obtained over a long period of time.

In the subculturing, part of the callus previously cultured may be cultured without modification, or microplasts prepared from part of the cultured callus may be suspension-cultured in a liquid medium. The conditions for performing the subculturing are preferably identical to the conditions described above for the induction of callus. The culturing period of one generation in the subculturing is preferably 5 to 30 days. The subculturing can be continued renewing generations unless the production of desired triterpenes in the callus decreases remarkably.

Among the triterpenes produced in the callus cultured as described above, corosolic acid and tormentic acid have high usefulness in respect of pharmacological actions, so the corosolic acid and tormentic acid content of the callus is preferably high. Specifically, the callus preferably contains maslinic acid, corosolic acid at 4-10 times by weight with respect to maslinic acid, tormentic acid at 2-6 times by weight, and 2α,19α-dihydroxy-3-oxo-urs-12-en-28-oic acid at 1.5-4 times by weight.

To facilitate obtaining corosolic acid, which has a strong hypoglycemic action, in high purity, it is important that the ratio of corosolic acid to maslinic acid in the callus is high. Corosolic acid and maslinic acid have very similar chemical structures, so it is difficult to separate them.

The triterpenes in the callus may be quantitatively determined by analyzing with liquid chromatography components extracted from the cultured callus using methanol etc., and calculating the area of the obtained chromatogram.

An extract which is extracted from the cultured callus by methods known in the art is a triterpene composition containing large amounts of corosolic acid, tormentic acid, 2α,19α-dihydroxy-3-oxo-urs-12-en-28-oic acid and maslinic acid. This triterpene composition may also be used as an ingredient of health foods and the like, and may be further purified to remove unnecessary components.

The extraction from the cultured callus may be performed using the raw callus after the culturing, or the callus may be used after drying and crushing.

The solvent used for extraction of the triterpenes is preferably a hydrophilic solvent, for example water or an alcohol such as methanol or ethanol, but more preferably, a warm water/alcohol mixed solvent. Specifically, a suitable method is one wherein ethanol or an aqueous ethanol solution (50-80 wt % ethanol content) is added to dried pulverized banaba leaves (raw material) at 5-20 times by weight, preferably 8-10 times by weight, with respect to the raw material, the mixture is heated to reflux for extraction at a temperature from normal temperature to 90° C., preferably from about 50° C. to 85° C., for a period from 30 minutes to 2 hours, and the extraction is repeated 2 or 3 times.

From the triterpene composition thus obtained, the individual triterpenes can also be obtained by isolating and purifying corosolic acid, tormentic acid, 2α,19α-dihydroxy-3-oxo-urs-12-en-28-oic acid or maslinic acid. These triterpenes can be isolated and purified, for example, using silica gel column chromatography or by recrystallization.

When there is a large amount of the triterpene composition, the following method is preferred. After suspending the triterpene composition in water, it is distributed in ether or hexane to first remove low polarity components. The aqueous layer is then successively eluted with water, methanol and acetone using Diaion HP-20 column chromatography or the like. The methanol fraction is then subjected to separation and purification using silica gel column chromatography or the like.

When trying to obtain the triterpenes in high purity by separation and purification, the following method is also preferred. After acetylation of hydroxyl groups and methyl esterification of carboxyl groups in the triterpenes, purification is performed using silica gel chromatography or by recrystallization, and hydrolysis is performed to obtain the desired triterpenes.

The corosolic acid, tormentic acid, 2α,19α-dihydroxy-3-oxo-urs-12-en-28-oic acid or maslinic acid obtained by the above isolation and purification contains at least 90.0 weight % or more, preferably 98.0 weight % or more, and more preferably 99.9 weight % or more, of the individual triterpene.

The triterpene composition of the present invention is obtained by the method described above, and contains at least one selected from the group consisting of corosolic acid, which is represented by the following formula (1), tormentic acid, which is represented by the following formula (2), 2α,19α-dihydroxy-3-oxo-urs-12-en-28-oic acid, which is represented by the following formula (3), and maslinic acid, which is represented by the following formula (4).

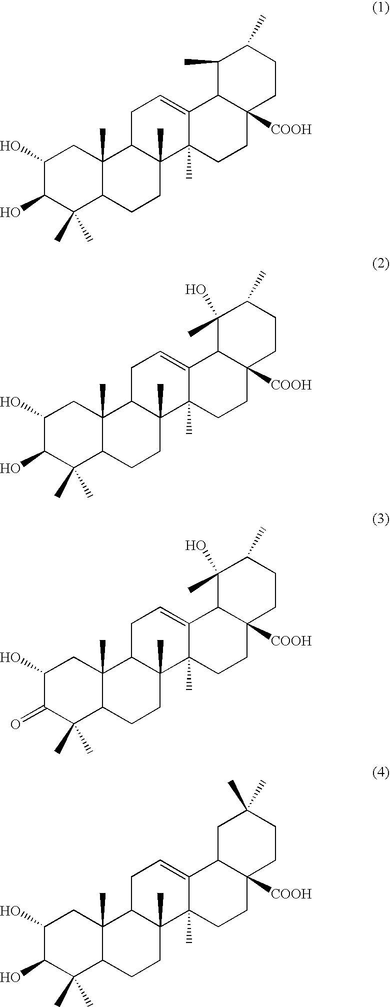

The triterpene composition containing these triterpenes has pharmacological actions such as a blood glucose depressant action and an antitumor action, and can be used as an ingredient of health foods and medicines. Triterpene compositions containing corosolic acid and tormentic acid which have strong pharmacological actions are particularly useful.

### EXAMPLES

The present invention will now be explained in greater detail referring to the following examples, with the understanding that these examples are in no way limitative on the present invention.

(Induction of Callus)

A leaf cut from banaba was immersed in sodium hypochlorite for 15 minutes, and sterilization treatment was performed. 10 μM of indoleacetic acid and 10 μM of kinetin were added as plant hormones to an LS agar medium in an Erlenmeyer flask, the medium was adjusted to pH 5.8, the sterilized banaba leaf was placed therein, and callus was induced by cultivating the leaf at 25° C. in a dark place.

After cultivating the induced callus for 30 days, part of the callus was transplanted to another LS agar medium and cultured, and it was found that the transplanted callus increased again. In other words, it was possible to perform the subculturing of callus induced from banaba.

(Callus Culturing, and Triterpene Content of Callus)

The induced callus was cultured for three weeks and proliferated in the LS agar medium to which 10 μM of indoleacetic acid and 10 μM of kinetin had been added. The proliferated callus was immersed in cold methanol, and a chromatogram was obtained by conducting high performance liquid chromatography analysis (ultraviolet absorptiometer, detection wavelength: 220 nm) of the extracted components. The triterpene content of the cultured callus was determined from the area of the peak corresponding to each triterpene. The triterpene content of the original plant (banaba leaf) before the induction of callus was also determined by the same method. The results are shown in Table 1.

TABLE 1

| | Callus | | Original plant | |
|---|---|---|---|---|
| | Content | Weight | Content | Weight |
| Triterpene | [mg/g dry wt.] | ratio | [mg/g dry wt.] | ratio |
| Corosolic acid | 10.7 | 6.7 | 1.1 | 5.5 |
| Tormentic acid | 6.0 | 3.8 | Not detected | — |
| 2α,19α-dihydroxy- | 3.3 | 2.1 | Not detected | — |
| 3-oxo-urs-12-en- | | | | |
| 28-oic acid | | | | |
| Ursolic acid | 0.5 | 0.8 | 0.2 | 1.0 |
| Oleanolic acid | 0.4 | 0.3 | 0.1 | 0.5 |
| Maslinic acid | 1.6 | 1.0 | 0.2 | 1.0 |

The corosolic acid content of callus was about 10 times higher than in the original plant. It was also found that tormentic acid and 2α,19α-dihydroxy-3-oxo-urs-12-en-28-oic acid, which were not detected in the original plant, were produced when callus was induced.

It was also attempted to culture callus in an LS agar medium to which only indoleacetic acid or kinetin had been added as a plant hormone, and in an LS agar medium to which no plant hormone was added, but in these cases, effectively little proliferation of callus was seen.

According to the method of producing a triterpene composition of the present invention, a triterpene composition containing at least one selected from the group consisting of corosolic acid, tormentic acid, 2α,19α-dihydroxy-3-oxo-urs-12-en-28-oic acid and maslinic acid can be stably and efficiently supplied.

1. A method of producing a triterpene composition wherein callus induced from banaba, loquat, perilla or guava is cultured with indoleacetic acid and another plant hormone, and a triterpene composition containing at least one selected from the group consisting of corosolic acid, tormentic acid, 2α,19α-dihydroxy-3-oxo-urs-12-en-28-oic acid and maslinic acid is obtained from the cultured callus.

2. The method of producing a triterpene composition according to claim 1, wherein said culturing is subculturing of said callus.

3. The method of producing a triterpene composition according to claim 1, wherein said callus is callus induced from the leaves, seeds, stems, shoot tips, roots or germ cells of banaba, loquat, perilla or guava.

4. The method of producing a triterpene composition according to claim 1, wherein said cultured callus contains maslinic acid, corosolic acid at 4-10 times by weight with respect to maslinic acid, tormentic acid at 2-6 times by weight with respect to maslinic acid, and 2α,19α-dihydroxy-3-oxo-urs-12-en-28-oic acid at 1.5-4 times by weight with respect to maslinic acid.

5. A triterpene composition obtained by the method of producing a triterpene composition according to claim 1, wherein said triterpene composition contains at least one selected from the group consisting of corosolic acid, tormentic acid, 2α,19α-dihydroxy-3-oxo-urs-12-en-28-oic acid and maslinic acid.

6. Callus induced from banaba, loquat, perilla or guava, wherein said callus is used in the method of producing a triterpene composition according to claim 1.

7. A method of producing corosolic acid wherein callus induced from banaba, loquat, perilla or guava is cultured with indoleacetic acid and another plant hormone, a triterpene composition containing corosolic acid is obtained from the cultured callus, and corosolic acid is obtained from the triterpene composition.

8. The method of producing corosolic acid according to claim 7, wherein said culturing is subculturing of callus.

9. The method of producing corosolic acid according to claim 7, wherein said callus is callus induced from the leaves, seeds, stems, shoot tips, roots or germ cells of banaba, loquat, perilla or guava.

10. A method of producing tormentic acid wherein callus induced from banaba, loquat, perilla or guava is cultured with indoleacetic acid and another plant hormone, a triterpene composition containing tormentic acid is obtained from the cultured callus, and tormentic acid is obtained from the triterpene composition.

11. The method of producing tormentic acid according to claim 10, wherein said culturing is subculturing of callus.

12. The method of producing tormentic acid according to claim 10, wherein said callus is callus induced from the leaves, seeds, stems, shoot tips, roots or germ cells of banaba, loquat, perilla or guava.

13. A method of producing 2α,19α-dihydroxy-3-oxo-urs-12-en-28-oic acid wherein callus induced from banaba, loquat, perilla or guava is cultured with indoleacetic acid and another plant hormone, a triterpene composition containing 2α,19α-dihydroxy-3-oxo-urs-12-en-28-oic acid is obtained from the cultured callus, and 2α,19α-dihydroxy-3-oxo-urs-12-en-28-oic acid is obtained from the triterpene composition.

14. The method of producing 2α,19α-dihydroxy-3-oxo-urs-12-en-28-oic acid according to claim 13, wherein said culturing is subculturing of callus.

15. The method of producing 2α,19α-dihydroxy-3-oxo-urs-12-en-28-oic acid according to claim 13, wherein said callus is callus induced from the leaves, seeds, stems, shoot tips, roots or germ cells of banaba, loquat, perilla or guava.

16. A method of producing maslinic acid wherein callus induced from banaba, loquat, perilla or guava is cultured with indoleacetic acid and another plant hormone, a triterpene composition containing maslinic acid is obtained from the cultured callus, and maslinic acid is obtained from the triterpene composition.

17. The method of producing maslinic acid according to claim 16, wherein said culturing is subculturing of callus.

18. The method of producing maslinic acid according to claim 16, wherein said callus is callus induced from the leaves, seeds, stems, shoot tips, roots or germ cells of banaba, loquat, perilla or guava.
